(19) United States
(12) Patent Application Publication
(10) Pub. No.: US 2024/0238621 A1
(43) Pub. Date: Jul. 18, 2024

(54) HEAD FOR ULTRASOUND THERAPY

(71) Applicant: EUNSUNG GLOBAL CORP., Gangwon-do (KR)

(72) Inventor: Ki Se LEE, Seoul (KR)

(21) Appl. No.: 18/515,330

(22) Filed: Nov. 21, 2023

(30) Foreign Application Priority Data
Jan. 13, 2023 (KR) 10-2023-0005389

(51) Int. Cl. A61N 7/02

(52) U.S. Cl. CPC A61N 7/02

(57) ABSTRACT

Proposed is a head for ultrasound therapy including an upper portion and a lower portion, wherein the lower portion includes a space portion and an ultrasound transducer portion moving inside the space portion that is filled with a coolant.

### CROSS REFERENCE TO RELATED APPLICATION

The present application claims priority to Korean Patent Application No. 10-2023-0005389, filed on Jan. 13, 2023, the entire contents of which are incorporated herein for all purposes by this reference.

### BACKGROUND

### Technical Field

Exemplary embodiments of the present disclosure relate to a head for ultrasound therapy.

### Description of the Related Art

High-intensity focused ultrasound (HIFU) has been growing in popularity and has gained support as a therapeutic device in the medical industry. Ultrasound energy has been extensively used in non-therapeutic procedures such as tissue imaging for diagnosis. High-intensity focused ultrasound (HIFU) is used with higher levels of power (beyond diagnostic ultrasound) to achieve diverse physical effects on tissue with the goal of attaining a desired therapeutic effect.

The cyclic design issue for HIFU treatment devices is tailored between the needs of the therapeutic demands required in a treatment and the acceptability of therapeutic devices produced by manufacturers of medical devices. This is especially true for aesthetic healthcare, where devices with therapeutic usefulness have to meet strict utility, imaging, and usability demands of doctors for aesthetic medicine and customers.

### SUMMARY

A problem that the present disclosure seeks to solve is to provide a head for ultrasound therapy with a more advanced structure.

Problems of the present disclosure are not limited to those mentioned above, and other problems not mentioned will be apparent to those skilled in the art from the following description.

In order to solve the problem above, a head for ultrasound therapy according to an exemplary embodiment of the present disclosure includes an upper portion and a lower portion, and the lower portion includes a space portion and an ultrasound transducer portion moving inside the space portion wherein the space portion is filled with a coolant.

In a head for ultrasound therapy according to an exemplary embodiment of the present disclosure, an ultrasound transmission portion may be disposed in the lower portion.

In a head for ultrasound therapy according to an exemplary embodiment of the present disclosure, the coolant is supplied by a coolant supply portion, and the coolant supply portion may be disposed in an upper and lower compartment portion.

In a head for ultrasound therapy according to an exemplary embodiment of the present disclosure, the coolant supply portion includes a coolant spray portion disposed inside the space portion, and the coolant spray portion may spray a coolant into the interior of the space portion.

In a head for ultrasound therapy according to an exemplary embodiment of the present disclosure, the supply flow rate of the coolant may be continuously regulated when the head for ultrasound therapy operates.

In a head for ultrasound therapy according to an exemplary embodiment of the present disclosure, when a head for ultrasound therapy operates, the temperature of the head may be regulated by supplying a coolant into the space portion of the lower portion.

The effects of the present disclosure are not limited to those mentioned above, and other effects not mentioned will be apparent to those skilled in the art from the description of the claims.

### BRIEF DESCRIPTION OF THE DRAWINGS

FIG. 1 is a perspective view showing the exterior of a head for ultrasound therapy according to an exemplary embodiment of the present disclosure.

FIG. 2 is a view showing the interior of a head for ultrasound therapy according to an exemplary embodiment of the present disclosure.

FIG. 3 is a view showing the interior of a side surface portion of a head for ultrasound therapy according to an exemplary embodiment of the present disclosure.

FIG. 4 is a view showing the interior of a front portion of a head for ultrasound therapy according to an exemplary embodiment of the present disclosure.

FIG. 5 is a view showing the interior of an upper portion of a head for ultrasound therapy according to an exemplary embodiment of the present disclosure.

FIG. 6 is a view showing the interior of a lower portion of a head for ultrasound therapy according to an exemplary embodiment of the present disclosure.

### DETAILED DESCRIPTION OF THE DISCLOSURE

It should be understood that the exemplary embodiments described herein are merely exemplary in order to help the understanding of the present disclosure and that the present disclosure may be variously modified and differently implemented from the exemplary embodiments described herein. However, detailed descriptions and specific drawings are omitted when it is determined in describing the present disclosure that a detailed description of a related known function or component would unnecessarily obscure the gist of the present disclosure.

The first and second terms used in this application may be used to describe various components, but components should not be limited by the terms. The terms are used only to distinguish one component from another.

Furthermore, the terms used in this application are intended only to describe specific exemplary embodiments and are not intended to limit the scope of the claims. Singular expressions include plural expressions unless the context clearly indicates otherwise. In this application, the terms “includes”, “consists of”, “comprises” or the like are intended to designate the presence of features, numbers, steps, actions, components, parts, or combinations thereof described in the specification, and should be understood not to preclude the possibility of the presence or addition of one or more other features, numbers, steps, actions, components, parts, or combinations thereof in advance.

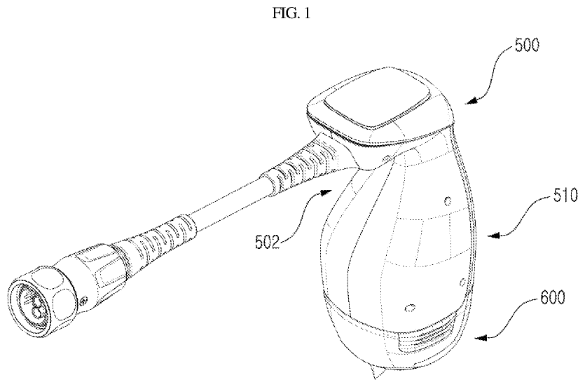

Hereinafter, referring to FIGS. 1 to 6, a head for ultrasound therapy according to an exemplary embodiment of the present disclosure will be described.

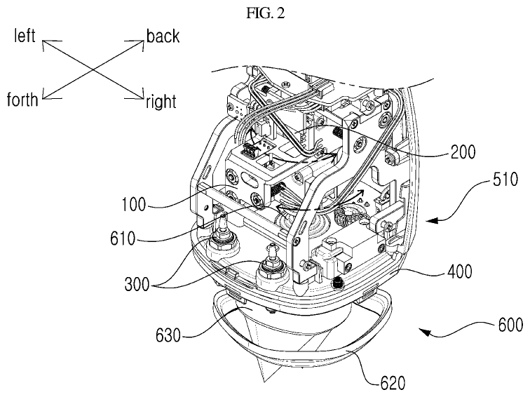

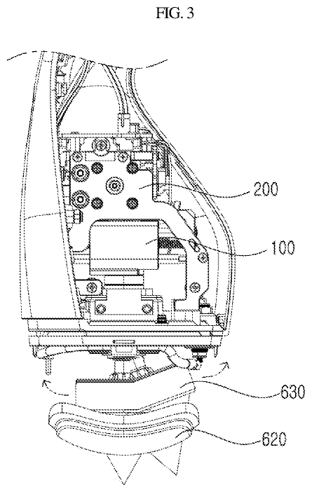

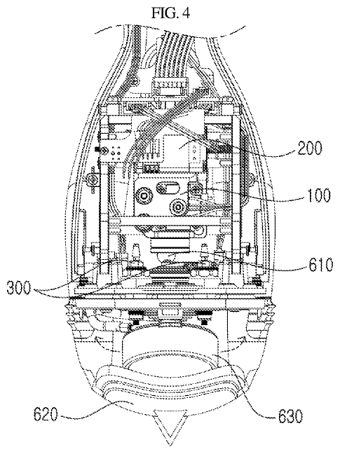

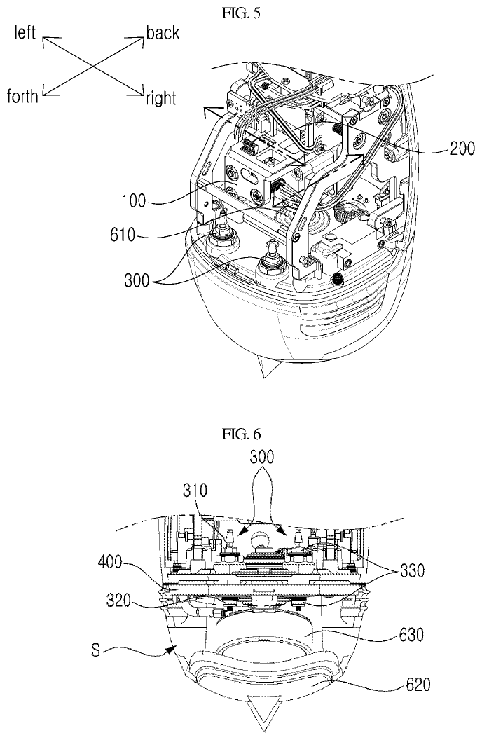

FIG. 1 is a perspective view showing the exterior of a head for ultrasound therapy according to an exemplary embodiment of the present disclosure. FIG. 2 is a view showing the interior of a head for ultrasound therapy according to an exemplary embodiment of the present disclosure. FIG. 3 is a view showing the interior of a side surface portion of a head for ultrasound therapy according to an exemplary embodiment of the present disclosure. FIG. 4 is a view showing the interior of a front portion of a head for ultrasound therapy according to an exemplary embodiment of the present disclosure. FIG. 5 is a view showing the interior of an upper portion of a head for ultrasound therapy according to an exemplary embodiment of the present disclosure. FIG. 6 is a view showing the interior of a lower portion of a head for ultrasound therapy according to an exemplary embodiment of the present disclosure.

Referring to FIGS. 1 to 6, a head 500 according to an exemplary embodiment of the present disclosure may include an upper portion 510 and a lower portion 600. The upper portion 510 may include a grip portion 502 which may be designed to be held by a left-handed or right-handed person. A bottom portion of the lower portion 600 includes an ultrasound transmission portion 620.

In general, the upper portion includes an actuator such as a motor driving device necessary to operate the head and various electronic devices. The lower portion includes an ultrasound transducer portion 630. The lower portion 600 is in a wet environment where fluid surrounds the ultrasound transducer portion 630 so that an ultrasound coupling medium is provided between the ultrasound transducer portion 630 and the ultrasound transmission portion 620.

The fluid in the lower portion 600 may be a fluid or a low molecular weight solution having the characteristics of a high thermal mass with a low ultrasound impedance. The fluid may be, for example, water. The water may be water free of impurities, including degassed non-ionized water, degassed and distilled water, or degassed and filtered water. The lower portion 600 is attachable and detachable as a form of a cartridge.

The ultrasound transducer portion 630 may be a high-intensity focused ultrasound (HIFU) transducer. Therapeutic ultrasound may be provided to a human body through a high-intensity focused ultrasound (HIFU) transducer. Ultrasound waves provided through the ultrasound transducer portion 630 may enable the human body contouring by being transmitted to the human body through the ultrasound transmission portion 620.

When operating a head for ultrasound therapy, a technician may activate a medical ultrasound system adjacent to a human body (patient). The technician may move the head 500 while gripping it. At this time, the head 500 may be aligned such that the ultrasound transmission portion 620 comes into contact with a patient. A user interface device may be operated to perform appropriate treatments or diagnostic tests. In this case, the ultrasound transducer portion 630 mounted on the lower portion 600 may generate ultrasound energy and may be used, for example, in order to destroy fatty tissue in a patient.

In addition to the ultrasound transducer portion 630, the lower portion 600 may include a fluid, such as water, used to combine with ultrasound energy passing through the ultrasound transmission portion 620 from the ultrasound transducer portion 630 to an ultrasound transmission portion 620 located near the bottom of the lower portion 600.

A drive portion including a motor may be disposed in the upper portion 510. More specifically, the upper portion 510 may include a first axial movement portion 100 and a second axial movement portion 200. The first axial movement portion may move in a back and forth direction. The second axial movement portion 200 may move in a left and right direction with the first axial movement portion 100. The first axial movement portion 100 and the second axial movement portion 200 may include a motor.

The first axial movement portion 100 may be connected to a ball joint 610. The ball joint 610 may pass through an upper and lower compartment portion 400 and be connected to the ultrasound transducer portion 630. Accordingly, as the first axial movement portion 100 and the second axial movement portion 200 move, the ultrasound transducer portion 630 connected to them may move in back and forth and left and right directions with the ball joint 610 as the center of rotation.

The ultrasound transmission portion 620 may be formed in a gently curved shape with a shape raised toward the outside. The ultrasound transducer portion 630 may move in back and forth and left and right directions via the ball joint 610 in a path similar to pendulum movements in order to correspond to the gently curved inner surface of the ultrasound transmission portion 620.

Through this, the direction of ultrasound waves provided by the ultrasound transducer portion 630 may be determined according to a situation, the ultrasound transducer portion 630 emits ultrasound energy while moving along a desired movement path and ultrasound energy is emitted along a predetermined movement path while passing through the ultrasound transmission portion 620 so that ultrasound waves are emitted to a human body along a emission movement path of ultrasound energy.

The ultrasound transmission portion 620 is formed in a lower part of the lower portion 600 of the head 500 and may be a part that comes into contact with a human body when a head for the ultrasound therapy operates.

The space portion S of the lower portion 600 may be supplied with a coolant. The coolant may be supplied by a coolant supply portion 310, and the coolant supply portion 310 may be installed in the upper and lower compartment portion 400. The coolant supply portion 310 may include a coolant spray portion 320 disposed inside the space portion S, and the coolant spray portion 320 may spray a coolant into the interior space of the space portion S.

Next, a coolant recovery portion 330 that absorbs and recovers a coolant in a state where the interior of the space portion S is filled with the coolant sprayed by the coolant spray portion 320 may be installed in the upper and lower compartment portion 400. The coolant recovery portion 330 and the coolant supply portion 310 may be disposed and spaced from each other at a certain distance and heat exchange is easily possible without an additional configuration for heat exchange by recovering the coolant wherein heat has been exchanged by the coolant spray portion 320 in the space portion S.

The ultrasound transducer portion 630 may move in back and forth and left and right directions inside the space portion S. When a head for ultrasound therapy operates, a coolant may be continuously supplied to the space portion S.

When a head for the ultrasound therapy operates, the temperature inside the lower portion 600 may rise due to the driving of the ultrasound transducer portion 630 and the driving of the first and second axial movement portions 100, 200 of the upper portion as well as a motor. At this time, the temperature of the lower portion 600 is regulated by supplying a coolant into the interior of the space portion S. As the temperature of the fluid inside the space portion S increases, the supply flow rate of the coolant may be continuously regulated so that the temperature of the fluid inside the space portion S may be regulated.

Although preferred exemplary embodiments of the present disclosure have been illustrated and described above, the present disclosure is not limited to the specific exemplary embodiments described above, and various modifications may be made by those skilled in the art without departing from the gist of the present disclosure as claimed in the claims and such exemplary embodiments should not be individually understood from the technical spirit or prospect of the present disclosure.

What is claimed is:

1. A head for ultrasound therapy, the head comprising:
an upper portion; and
a lower portion, wherein the lower portion comprises:
a space portion; and
an ultrasound transducer portion moving inside the space portion wherein the space portion is filled with a coolant.

2. The head for ultrasound therapy of claim 1, wherein an ultrasound transmission portion is disposed in the lower portion.

3. The head for ultrasound therapy of claim 1, wherein the coolant is supplied by a coolant supply portion and the coolant supply portion is disposed in an upper and lower compartment portion between the upper and lower portions.

4. The head for ultrasound therapy of claim 3, wherein the coolant supply portion comprises a coolant spray portion disposed inside the space portion, and the coolant spray portion sprays a coolant into an interior of the space portion.

5. The head for ultrasound therapy of claim 1, wherein the ultrasound transducer portion moves in back and forth and left and right directions inside the space portion.

6. The head for ultrasound therapy of claim 1, wherein a supply flow rate of a coolant is continuously regulated when the head for ultrasound therapy operates.
